(19) United States
(12) Patent Application Publication
(10) Pub. No.: US 2024/0307653 A1
(43) Pub. Date: Sep. 19, 2024

(54) Method and Apparatus for Mitigating Emotional or Physical Tension

(71) Applicant: S-ALPHA THERAPEUTICS, INC., Seoul (KR)

(72) Inventors: Myoung Joon KIM, Seoul (KR); Yong Han KIM, Gyeonggi-do (KR); Seung Eun CHOI, Seoul (KR); Ja Rang HAHM, Seoul (KR)

(73) Assignee: S-ALPHA THERAPEUTICS, INC., Seoul (KR)

(21) Appl. No.: 18/270,289

(22) PCT Filed: Nov. 3, 2021
(86) PCT No.: PCT/KR2021/015831

§ 371 Date: Jun. 29, 2023

Related U.S. Application Data
(60) Provisional application No. 63/109,010, filed on Nov. 3, 2020.

(51) Int. Cl. A61M 21/02; A61M 21/00; G16H 20/70

(52) U.S. Cl. CPC A61M 21/02; G16H 20/70; A61M 2021/005; A61M 2205/3303

(57) ABSTRACT

Methods and devices for mitigating emotional and physical tension or stress before, during or after a medical procedure. The present disclosure also provides methods for providing a series of visual and/or auditory inputs provided to a subject, wherein subsequent inputs are determined based on feedback from the subject following earlier inputs.

This application claims the benefit of and priority to U.S. Provisional Patent Application No. 63/109,010, filed on Nov. 3, 2020, the entirety of which is hereby incorporated by reference.

### TECHNICAL FIELD

Methods and devices for mitigating emotional or physical tension or stress before, during or after a medical procedure. The present disclosure also provides methods for providing a series of visual and/or auditory inputs provided to a subject, wherein subsequent inputs are determined based on feedback from the subject following earlier inputs.

### BACKGROUND ART

The sights, sounds, and tactile sensations of medical procedures can cause anxiety and stress in patients. For example, during ophthalmic surgery, following the administration of a local anesthetic, patients often hear the sound the procedure being performed on their eye. This leads to anxiety and stress in the patient, which in turn can distract the healthcare provider, adversely affect the success of the medical procedure, and/or delay patient recovery. Accordingly, there is a need for methods and devices for mitigating emotional or physical tension or stress before, during or after a medical procedure.

### DISCLOSURE OF INVENTION

### Solution to Problem

Methods and devices for mitigating emotional and physical tension or stress before, during or after a medical procedure. The present disclosure also provides methods for providing a series of visual and/or auditory inputs provided to a subject, wherein subsequent inputs are determined based on feedback from the subject following earlier inputs.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF DESCRIPTION OF DRAWINGS

FIG. 1A is a flow diagram of a subject's sensory points (labeled 1-6) can be affected by various sights and sounds of the surgery (labeled I-V), resulting in one or more uncomfortable physical stimulation(s) in the subject (labeled A-D);

FIG. 1B shows various strategies for distracting the subject or interfering with the subject's sensory points to perceive the various sights and sounds of the surgery. The blocking strategies are overlayed onto the flow diagram of FIG. 1A to show which blocking strategies may be used to block each sensory input;

FIG. 2 is a block diagram of an exemplary computing module of the present disclosure for use in mitigating emotional or physical tension in a subject during a medical procedure;

FIG. 3 is a block diagram of an exemplary system of the present disclosure for use in mitigating emotional or physical tension in a subject during a medical procedure; and

FIG. 4, items A-C show various visual stimulation, including (A) an image (e.g., a face), (B) an image in which a portion is in focus and a portion is out of focus, and (C) an object (e.g., a shape), which the subject should follow with their eye(s), and the corresponding direction (double headed arrows) in which the subject's eye(s) should move when viewing the visual stimulation is shown below each frame.

FIG. 5, items (A) and (B), show an example device for replacement sensory input.

FIG. 6, items (A) and (B), show another example device for replacement sensory input.

FIG. 7 shows a conceptual diagram of an example digital treatment device platform.

FIG. 8 shows a method for at-home treatment of anxiety according to an example embodiment.

FIG. 9 shows an example process for generating a customized prediction model.

FIG. 10 shows an example system for at-home treatment.

FIG. 11 shows an example system for emergent treatment.

FIG. 12 shows an example system for treatment during surgery.

FIG. 13 shows an example system for treatment and training for anxiety or a panic attack.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### MODE FOR THE INVENTION

### Definitions

As used herein, the term “stress” or “tension” refers to any physical, mental, or emotional factor that causes bodily or mental discomfort. Stress may manifest physically in a patient and thus may be measured or quantified in a variety of ways that are known in the medical field. Additionally or alternatively, stress may be measured through the information provided directly from a patient (e.g., patient surveys).

As used herein, the terms “treat”, “mitigate”, and “alleviate” are interchangeably used to mean a reduction in the occurrence of stress (e.g., emotional and/or physical tension) or of a symptom of stress. Thus, mitigating includes some reduction, significant reduction, near total reduction, and total reduction. An mitigating effect may appear immediately, or it not appear clinically for minutes, hours, or days after a medical procedure.

As used herein, the term “facial expression,” as used herein, refers to a facial output that results from the summation of individual facial muscles being in a contracted or relaxed state at a given point in time.

As used herein, the term “medical procedure” includes but is not limited to diagnostic procedures such as in vivo imaging, taking biopsies, surgical procedures and therapeutic procedures such as ablation, laser treatments, ultrasonic treatments, brachytherapy, and the like.

As used herein, the term “subject” may refer to a patient treated by any of the methods described herein or treated with a system or device described herein. A subject may be of any age and may be an adult, infant or child. In some cases, the patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). The patient may be a human or non-human subject. Any of the methods described herein may be performed on a non-human subject, such as a laboratory or farm animal. Non-limiting examples of a non-human subject include laboratory or research animals, a dog, a cat, or a non-human primate (e.g., a gorilla, an ape, an orangutan, a lemur, or a baboon).

As used herein, the singular terms “a,” “an,” and “the” include plural referents unless the context clearly indicates otherwise. Similarly, the word “or” is intended to include “and” unless the context clearly indicates otherwise.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary unless a particular order is clearly indicated by the context.

As used herein, the term “about” refers to a numeric value, including, for example, whole numbers, fractions, and percentages, whether or not explicitly indicated. The term “about” generally refers to a range of numerical values (e.g., +/−5, 6, 7, 8, 9 or 10% of the recited value) that one of ordinary skill in the art would consider equivalent to the recited value (e.g., having the same function or result). In some instances, the term “about” may include numerical values that are rounded to the nearest significant figure.

As used herein, the term “substantially” means the qualitative condition of exhibiting total or near-total extent or degree of a characteristic or property of interest. In some embodiments, “substantially” means within about 20%. In some embodiments, “substantially” means within about 15%. In some embodiments, “substantially” means within about 10%. In some embodiments, “substantially” means within about 5%.

### Method and Apparatus

In certain aspects, the present disclosure provides a method of mitigating stress in a subject. Stress may generally refer to any physical, mental, or emotional factor that causes bodily or mental discomfort. Stress may manifest physically in a patient and thus may be measured or quantified in a variety of ways that are known in the medical field (e.g., detecting sudden motion, a pattern of movement, perspiration, facial expressions, or the like). Additionally or alternatively, stress may be measured through the information provided directly from a patient (e.g., patient surveys). In certain aspects, the present disclosure provides a method of mitigating emotional or physical tension in a subject. Emotional tension in a subject may refer to, for example, an uncomfortable and unjustified sense of apprehension that may be diffuse and unfocused and is often accompanied by physiological symptoms (e.g., physical tension).

In certain embodiments, the present disclosure provides a method of mitigating emotional or physical tension in a subject during a medical procedure. In some embodiments, the present disclosure provides method of mitigating emotional or physical tension in a subject during a medical procedure involving an eye (e.g., a first eye and/or a second eye) of the subject. Deterioration or impairment of vision can occurs through aging, injury, irritation, and many unknown factors related to genetics. Of course, vision improvements are routinely achieved through the use of corrective lenses and by various medical procedures. Laser in-situ keratomileusis (LASIK surgery), involves surgical creation of a flap across the central portion of the cornea, and beneath this epithelial flap reshaping the cornea using a laser to improve the way light passes through the eye. Once the reshaping is complete, the flap is set down to heal. LASIK reshapes the underlying corneal stroma but does not modify the epithelium, excepting any residual ridges from the flap interface healing. Photorefractive keratectomy surgery (PRK) is used to correct mild to moderate nearsightedness, farsightedness, and/or astigmatism. During PRK surgery, the epithelium is fully abraded and removed prior to an excimer laser ablation of the stroma, followed by a healing period where the epithelium is encouraged to grow back over the reshaped stomal surface. In laser epithelial keratomileusis (LASEK), an epithelial flap is created by loosening epithelial cells using an alcohol solution, pulling the flap back prior to excimer reshaping of the stroma, then replacing and securing the flap with a soft contact lens while it heals. Cataract surgery involves replacement of a patient's cloudy natural lens with a synthetic lens to restore the lens's transparency. Refractive lens exchange (RLE) is similar to cataract surgery and involves making a small incision at the edge of the cornea to remove the natural lens of the eye and replacing it with a silicone or plastic lens. Presbyopic lens exchange (PRELEX), is a procedure in which a multifocal lens is implanted to correct presbyopia, a condition in which the eye's lens loses its flexibility.

In some embodiments, the methods of the present disclosure comprise providing to the subject, by an electronic device, a first unilateral visual input to a second eye of the subject; sensing, by a sensor connected to the electronic device, the emotional or physical tension in the subject; and based on the emotional or physical tension in the subject, providing a second unilateral visual input to the second eye of the subject to mitigate the emotional or physical tension in the subject.

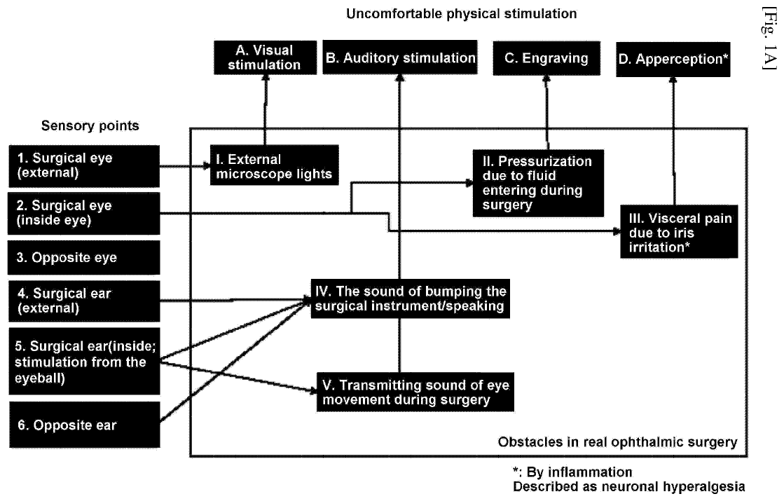

A visual stimulus or visual input provided to the eye not involved in the medical procedure can be used, for example, to reduce or mitigate emotional and/or physical tension in the subject by distracting the subject from the sights and sounds of the surgery (e.g., by immersing the subject in a comfortable or relaxing environment using physical, visual and/or auditory stimuli). FIG. 1 is a flow diagram of how various inputs (e.g., visual stimulation) may be used in various embodiments of the present disclosure for mitigating emotional or physical tension in a subject during a medical procedure. As shown in FIG. 1, comfort-inducing stimuli can be provided to the subject before, during, and/or after the medical procedure. For example, a visual stimulus can be used to distract the subject from external microscope lights used in the medical procedure. In another example, auditory stimuli can be used to generate interference or offset the sound of a healthcare provider coming into contact with a medical device or instrument used in the medical procedure. In yet another example, prior to and/or after the medical procedure, during an adaptation time or a recovery time, respectively, the first eye (e.g., the eye undergoing the medical procedure) or the second eye of the subject may be exercised using the electronic device (e.g., to loosen the eye muscles). In one embodiment, prior to the medical procedure, during an adaptation time, the first eye (e.g., the eye undergoing the medical procedure may be exercised using the electronic device (e.g., to loosen the eye muscles). In one embodiment, after the medical procedure, during a recovery time, the first eye (e.g., the eye undergoing the medical procedure may be exercised using the electronic device (e.g., for recovery or restoring a normal state of the eye). The adaptation time may be about 1 minute (min), about 5 min, about 10 min, about 15 min, about 20 min, about 25 min, about 30 min, about 35 min, about 40 min, about 45 min, about 50 min, about 55 min, about 60 min, about 70 min, about 80 min, about 90 min, about 120 min, or a range of any two values thereof. The recovery time may be about 1 min, about 5 min, about 10 min, about 15 min, about 20 min, about 25 min, about 30 min, about 35 min, about 40 min, about 45 min, about 50 min, about 55 min, about 60 min, about 70 min, about 80 min, about 90 min, about 120 min, or a range of any two values thereof. For example, the adaptation time may be between about 10 minutes and about 1 hour (i.e., 60 minutes). In another example, the recovery time may be between about 10 minutes and about 1 hour (i.e., 60 minutes). In some embodiments, ‘prior to a medical procedure’ may refer to a preoperative period of time (e.g., an adaptation period for the first eye or the second eye to a target). In some embodiments, ‘after a medical procedure’ may refer to a post-surgical period of time (e.g., a recovery period for the first eye or the second eye).

Visual input or visual stimulus can refer to any virtual or non-virtual image including but not limited to a product, object, stimulus, and the like, that an individual may view with their eyes. In certain embodiments of the present disclosure, the first unilateral visual inputs and second unilateral visual inputs may be independently selected from the group consisting of (i) an image, (ii) a video, (iii) one or more instructions from a healthcare provider associated with the medical procedure for the subject to follow, and (iv) information associated with the medical procedure. In some embodiments, the visual input can be unilateral (e.g., provided to one eye). In some embodiments, the visual input can be bilateral (e.g., provided to both eyes).

Since both human eyes may track together and in the same direction, in one embodiment, it is contemplated that the visual stimulus provided to a second eye of the subject may be used to direct motion in the first eye of the subject. For example, during a medical procedure, a healthcare provider may require the subject to suddenly or gradually move their eye (e.g., the eye on which the medical procedure is being performed) in a given direction. A visual input including an illustrated figure and instructions to follow the figure as it moves across the display may be provided to the subject's second eye. The illustrated figure can be programmed to move (e.g., with a given speed and direction) in a direction that the healthcare provider may require the subject's first eye to move for the medical procedure.

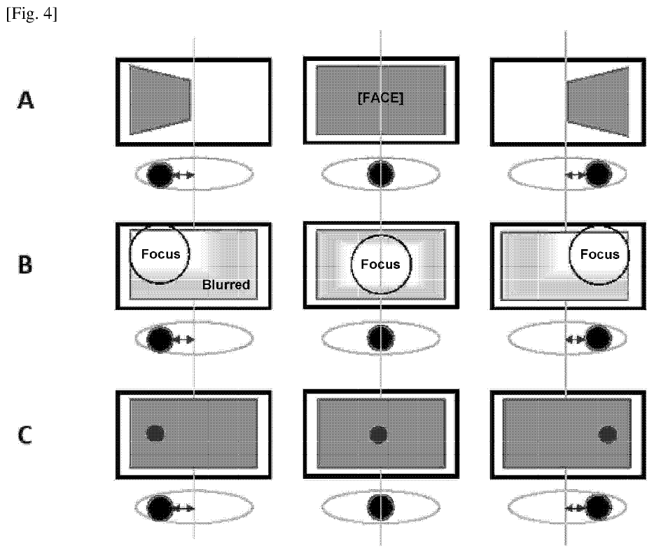

FIG. 4, items A-C show various visual stimulation that can be used with embodiments of the present disclosure, including (A) an image (e.g., a face), (B) an image in which a portion is in focus and a portion is out of focus, and (C) an object (e.g., a shape), which the subject should follow with their eye(s), and the corresponding direction (double headed arrows) in which the subject's eye(s) should move when viewing the visual stimulation is shown below each frame. In certain embodiments, the visual stimulation can comprise an object, and the object can be moved digitally in 1 dimension, 2 dimensions, or 3 dimensions. The subject may be instructed to follow the object with their eyes,

The electronic device may comprise, in some embodiments, one or more light sources or a display. In some embodiments comprising the display, the image, the video, the one or more instructions from a healthcare provider associated with the medical procedure for the subject to follow, and/or the information associated with the medical procedure can be provided to the subject using the display. The visual input, for example, can be an image or video that induces relaxation in the subject or distracts the subject from the upcoming or ongoing medical procedure. The content of the image may be age-specific. For example, a visual input for a young subject may be an image of a cartoon character, or an animated video. In another example, a visual input for an adult may be puzzle (e.g., a rebus puzzle), television (e.g., live television), or an immersive experience (e.g., on the beach). Of course, a visual input (e.g., such as an immersive experience) can also be supplemented with an auditory input.

In some embodiments, the visual input and/or the auditory input can also comprise instructions from a healthcare provider associated with the medical procedure for the subject to follow, and/or the information associated with the medical procedure. For example, instructions from the healthcare provider can include instructions for moving the subject's eyes or other body part in a specified direction. In another example, instructions from the healthcare provider can include instructions to hold still for a given period of time. In another example, instructions from the healthcare provider can include instructions to follow an object on the display for a given period of time. As discussed above, the tracking of an object on the display with the subject's second eye can be used to control the movement of the subject's first eye that is undergoing a medical procedure. In yet another example, the information associated with the medical procedure can be provided to the subject as a visual input (e.g., using the display) or as an auditory input (e.g., using a speaker), and the information can be, for example, the amount of time left for the medical procedure, the time until the start of the medical procedure, the stage of the medical procedure, information about the healthcare provider, and the like.

In some embodiments, the auditory input can comprise noise cancellation to minimize the sound heard by the subject from the healthcare provider or environment (e.g., operating room). The term noise cancellation or noise control is conventionally used to describe the process of minimizing or eliminating sound emissions from sources that interfere with the listeners' intended audio source, to increase comfort and relaxation. Conventionally, attempts at noise control and cancellation are performed via active or passive means. Active noise control is used to offset sound using a power source. Passive noise control refers to sound control by noise-reduction materials, such as insulations and sound-absorbing materials, and the like, rather than a power source. Active noise canceling can be useful for low frequency noise. However, as the target frequencies intended to be reduced become higher, the spacing requirements for free space and zone of silence techniques become prohibitive. This is mostly because the number of modes grows rapidly with increasing frequency, which quickly makes active noise control techniques unmanageable. Therefore, at such higher frequencies, passive treatments become more effective and often provide an adequate solution without the need for active control.

It is also contemplated that an electronic device of the present disclosure be operable to provide a unilateral visual input to either the left eye or the right eye of the subject. Accordingly, in some embodiments, the one or more light sources or the display can be releasably connected to the electronic device in a first position to interact with a right eye of the subject, or a second position to interact with a left eye of the subject. In some embodiments, the one or more light sources or the display can be releasably connected to the electronic device in a first position to interact with a left eye of the subject, or a second position to interact with a right eye of the subject. In some embodiments, the one or more light sources or the display can be releasably connected to the electronic device in a first position to interact with a right eye of the subject, or a second position to interact with a left eye of the subject. In other embodiments, the one or more light sources or the display are slidably connected to the electronic device, wherein the one or more light sources or the display can slide between a first position to interact with a right eye of the subject and a second position to interact with a left eye of the subject. In yet another embodiment, the one or more light sources or the display are slidably connected to the electronic device, wherein the one or more light sources or the display can slide between a first position to interact with a left eye of the subject and a second position to interact with a right eye of the subject. In another embodiment, the one or more light sources or the display are slidably connected to the electronic device, wherein the one or more light sources or the display can slide between a first position to interact with a right eye of the subject and a second position to interact with a left eye of the subject.

In some embodiments, the electronic device can comprise a wearable device. As used herein, the term “wearable device” may encompass devices that may be worn by a user (e.g., via an arm band, a wrist band, a chest strap, etc.), and devices that may be attached to a user's skin (e.g., via adhesive material). In some embodiments, the wearable device comprises one or more of a monocular head mounted display and speakers (e.g., headphones or a sound output device). In some implementations, a wearable device may include a sensor system capable of obtaining physiological data from the user's body, such as eye movement data, perspiration data, temperature data, respiration rate data, oxygen saturation data, blood glucose data, blood pressure data, heart rate data, etc.

In some embodiments, a method of the present disclosure comprises sensing, by a sensor connected to the electronic device, the emotional or physical tension in the subject. In some embodiments, the sensor comprises one or more of an eye movement sensor, a perspiration sensor, a temperature sensor, a respiration rate sensor, an oxygen saturation sensor, a blood glucose sensor, a blood pressure sensor, a heart rate sensor and/or electrodermograph, a hand-held pressure sensor, an eye gaze sensor, an eye muscle sensor, and a voice sensor. In some embodiments the sensor is within or physically attached to the electronic device. In other embodiments, the sensor is connected to the electronic device wirelessly

In some embodiments, a method of the present disclosure comprises sensing, by a sensor connected to the electronic device, the emotional or physical tension in the subject; and based on the emotional or physical tension in the subject, providing a second unilateral visual input to the second eye of the subject to mitigate the emotional or physical tension in the subject. For example, the sensor may be a voice sensor, and the second unilateral visual input is selected and provided based on a vocal feedback from the subject.

Sensors may be used to obtain information about the subject before, during and after the medical procedure. In some embodiments, methods of the present disclosure comprise determining one or more properties of the subject before, during, and/or after the medical procedure. In some embodiments, these properties may be associated with the subject's stress level, nervousness, or emotional or physical tension. In some embodiments, the properties can include, for example, one or more of eye movement, amount or rate of perspiration, temperature, respiration rate, oxygen saturation, blood glucose level, blood pressure, heart rate, direct feedback from the subject. Based on the one or more properties of the subject, methods of the present disclosure can comprise adjusting one or more aspects of the current medical procedure and/or an additional medical procedure on the subject. An additional medical procedure may be performed on the same eye as the first or previous medical procedure was performed on, or it may be performed on a different eye than the first or previous medical procedure was performed on. An additional medical procedure may comprise, for example, 1 additional medical procedure, 2 additional medical procedures, 3 additional medical procedures, 4 additional medical procedures, 5 additional medical procedures, 6 additional medical procedure, 7 additional medical procedures, 8 additional medical procedures, 9 additional medical procedures, or 10 additional medical procedures. Non-limiting examples of an additional medical procedure can comprise an eye surgery and an ear surgery. These one or more aspects of the additional procedure can be independently selected from the group consisting of the first unilateral visual stimulation, the second unilateral visual stimulation, or the auditory stimulation of the medical procedure. In some embodiments, the one or more aspects of the additional procedure can include, for example, the parameters of the device and/or one or more aspects of a performance of the healthcare provider. The parameters of the device can include, for example, the visual input and/or the audio inputs provided to the subject. The performance of the healthcare provider can include, for example, a parameter of the medical procedure (e.g., the duration) and/or the approach for the medical procedure (e.g., the surgical approach) or a strategy for performing the medical procedure. In some embodiments, the sensor may be used to sense a sound from device used in the medical procedure (e.g., a surgical device).

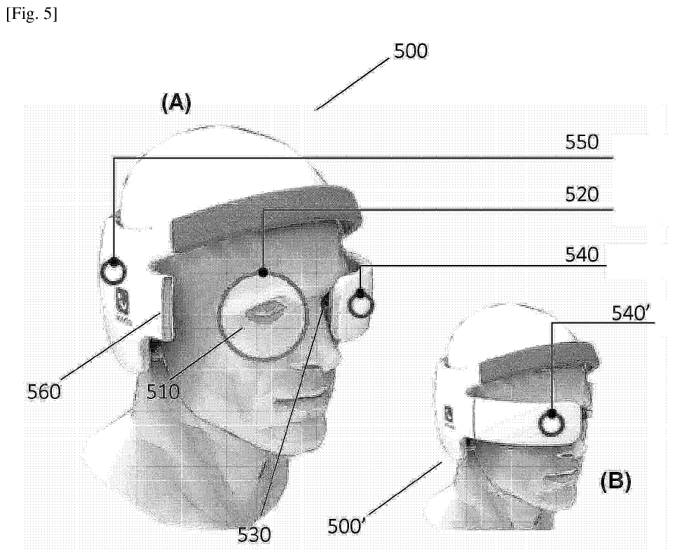

FIG. 5, items (A) and (B), show an example device for replacement sensory input. Some embodiments may provide suppression of visual information of the eye that is undergoing surgery. An image seen by the eye undergoing surgery may be perceived as being blurry or unfocused, as shown in FIG. 4, parts A through C, which may become a disturbing, or even terrifying, experience for the patient. However, in the surgical environment, bright light must be illuminated on the eye undergoing surgery, and the patient cannot prevent this image from entering the eye. By using the binocular suppression function, it is possible in some embodiments to suppress the vision that gives a feeling of fear to the eye undergoing surgery by presenting a clear stimulus perceived with the same brightness in the eye that is not undergoing surgery.

Some embodiments may provide distraction of the subject patient. For example, sound may be provided, e.g., providing beats of specific frequencies, to facilitate focus on sound and on a display screen. As another example, a display screen may be provided to keep the pupils of the patient from moving, while still providing a comfortable environment.

In the device 500 shown in FIG. 5, items (A) and (B), an eye undergoing surgery 510 is uncovered for surgical access. Item (A) shows an arbitrarily-chosen right-eye undergoing surgery first, and item (B) shows the other eye, e.g., a left eye, undergoing treatment. The selection of left vs. right eyes for treatment is arbitrary for the purposes of the present disclosure. A treatment area 520 is left open and uncovered to secure the field of vision for the operator, e.g., a surgeon. The other eye 530 that is not currently being treated may be covered by a closed-type monocular display 540. Sound may be provided at a sound output 550, e.g., a speaker, which may be integrated in some embodiments into the device 500. In other embodiments, a separate sound input may be provided, e.g., headphones, external speakers, earphones, and the like. The sound from the sound output 550 may be monaural, stereo sound, surround sound, or any other appropriate sound environment. In some embodiments, the monocular display 540 may be removeable and switchable to the other eye so that the other eye 530 may undergo surgery, as shown in FIG. 5, item (B) in which the device 500′ has the monocular display 540′ over the patient's right eye, rather than over the left eye in the device 500 with the monocular display 540 in FIG. 5, item (A). A port 560 may be provided in the device 500 into which the monocular display 540 may be inserted to receive images to display. The use of the switchable monocular display 540 avoids having to remove the entire device 500 from the patient's head and repositioning the surgical field in the middle of surgery, which would add time and risk complicating the surgery.

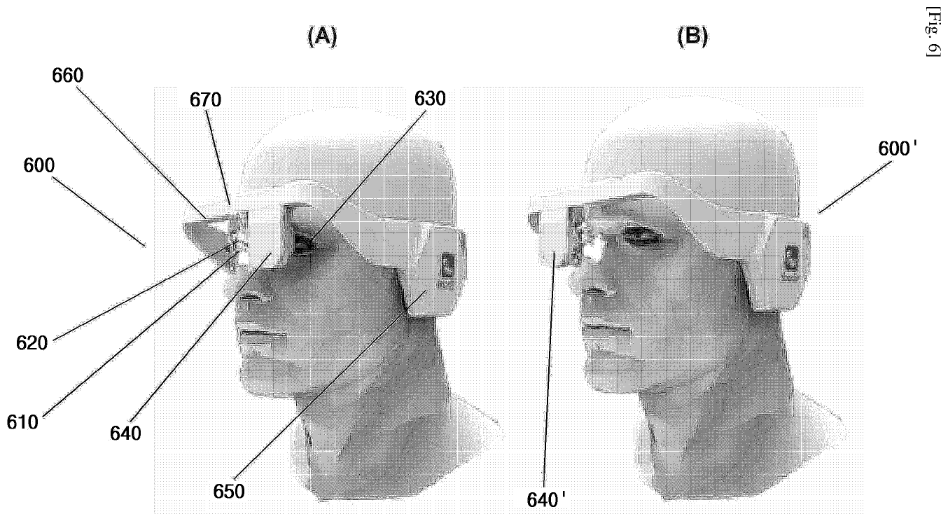

FIG. 6, items (A) and (B), show another example device for replacement sensory input. In the device 600 shown in FIG. 6, items (A) and (B), an eye undergoing surgery 610 is uncovered for surgical access. Item (A) shows an arbitrarily-chosen right-eye undergoing surgery first, and item (B) shows the other eye, e.g., a left eye, undergoing treatment. The selection of left vs. right eyes for treatment is arbitrary for the purposes of the present disclosure. A treatment area 620 is left open and uncovered to secure the field of vision for the operator, e.g., a surgeon. The other eye 630 that is not currently being treated may be covered by a closed-type monocular display 640 on a frame 670. Sound may be provided at a sound output 650, e.g., a speaker, which may be integrated in some embodiments into the device 600. In other embodiments, a separate sound input may be provided, e.g., headphones, external speakers, earphones, and the like. The sound from the sound output 650 may be monaural, stereo sound, surround sound, or any other appropriate sound environment. In some embodiments, the monocular display 640 may be removeable and switchable to the other eye so that the other eye 630 may undergo surgery, as shown in FIG. 6, item (B) in which the device 600′ has the monocular display 640′ over the patient's right eye, rather than over the left eye in the device 600 with the monocular display 640 in FIG. 5, item (A). A port 660 may be provided in the device 600 into which the monocular display 640 may be inserted to receive images to display. Alternatively, a track (not illustrated) may be provided to slide the monocular display 640 from one side of the patient's face to the other, to cover the other eye without removing the monocular display 640 completely. The use of the moveable monocular display 640 avoids having to remove the entire device 600 from the patient's head and repositioning the surgical field in the middle of surgery, which would add time and risk complicating the surgery.

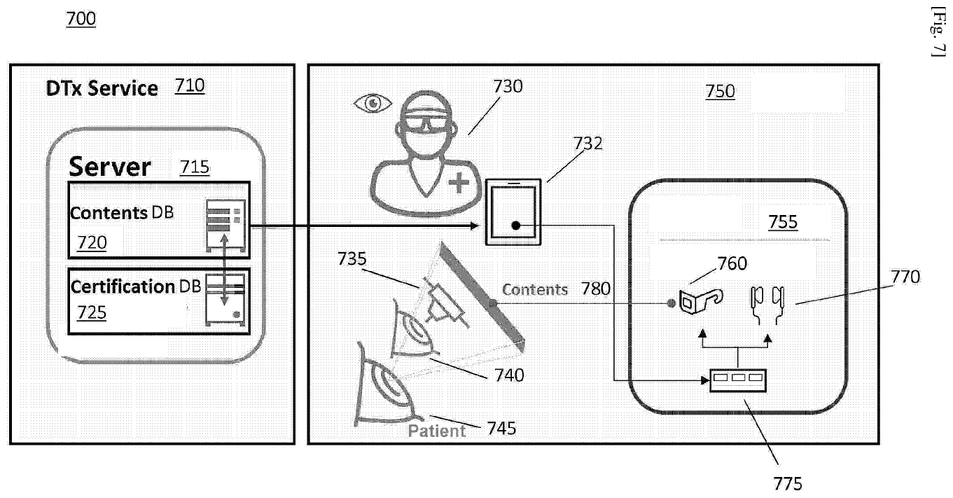

FIG. 7 shows a conceptual diagram of an example digital treatment device platform. The device platform 700 of FIG. 7 is an example of a digital therapy service to relieve panic attacks during an eye surgery procedure. The device platform 700 may enhance successful surgery, and may create additional revenue through digital treatment service for patients with fear and anxiety about the procedure. In the device platform 700, a data transmission (“DTx”) service 710 may include a server 715, which may include a contents database 720 and a certification database 725. The contents database 720 may store images to be transmitted to a surgical treatment location 750, e.g., a hospital, such that the contents may be viewed by a patient undergoing surgery. The certification database 725 may store certification data to create a secure transmission of information, including the image data, between the DTx service 710 and the surgical treatment location 750.

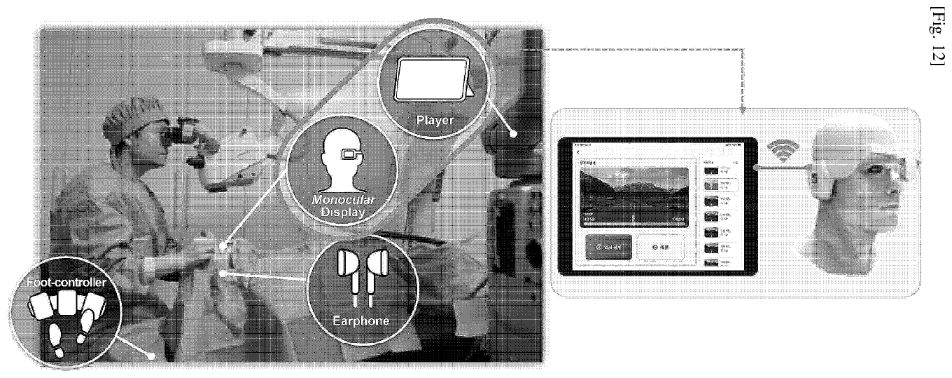

An operator 730, e.g., a surgeon, may receive the information from the DTx service 710, including contents 780 and sound data, which may include image data, on a control device 732. A device 755 may be positioned on the patient. The device 755 may include a monocular display 760 for providing the contents 780, including an image, to an eye not currently undergoing surgical treatment 745. The device 755 may further include a sound output 770 for providing sound to the patient. The eye currently undergoing surgical treatment 740 may have a surgical treatment device 735 in a surgical field. The device 755 may further include a content receiver for receiving the contents 780 and sound data from the control device 732, and respectively providing the contents 780 and sound data to the monocular display 760 and the sound output 770. The device 755 may also be controlled by a foot controller operable, e.g., by the control device 732, for example, as shown in FIG. 12.

The device 755 may be similar to the devices 500, 600 described above with reference to FIGS. 5-6. The monocular display 760 may be similar to the monocular displays 540, 640 described above with reference to FIGS. 5-6. The sound output 770 may be similar to the sound outputs 560, 660 described above with reference to FIGS. 5-6.

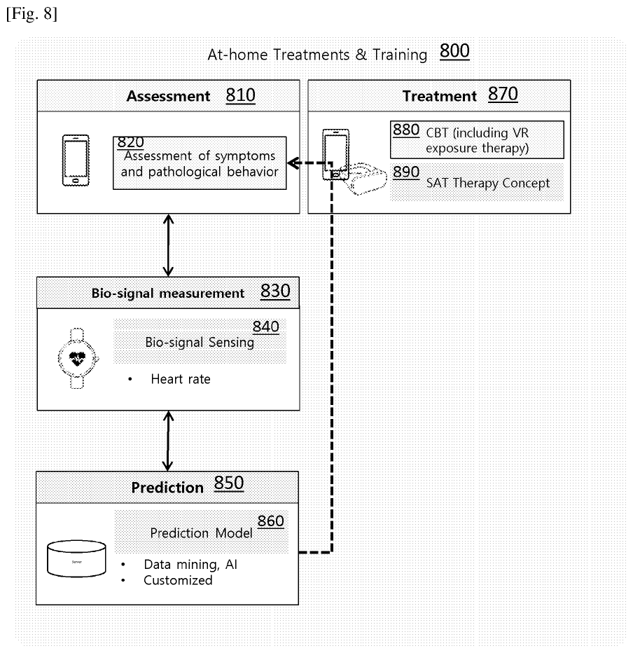

FIG. 8 shows a method for at-home treatment of anxiety according to an example embodiment. The term “at-home” as used herein, refers to activities outside of a medical setting. In the method 800, an assessment 810 may be performed. The assessment may include an assessment of symptoms and pathological behavior (820). The assessment 810 may include a bio-signal measurement 830, which may include, e.g., measuring heart rate, respiration, or other biological data. The bio-signal measurement 830 may be performed using a sensor in, for example, by a wearable device, such as a smart watch or activity tracker, or in another external device. The assessment 810 may also include a prediction 850, which may be made according to a prediction model 860. The prediction model 860 may be obtained through a database that may be provided by, for example, data mining, artificial intelligence (AI) or machine learning, or may be customized. The customization may be made, for example, with patient data, which may include the bio-signal measurement 830.

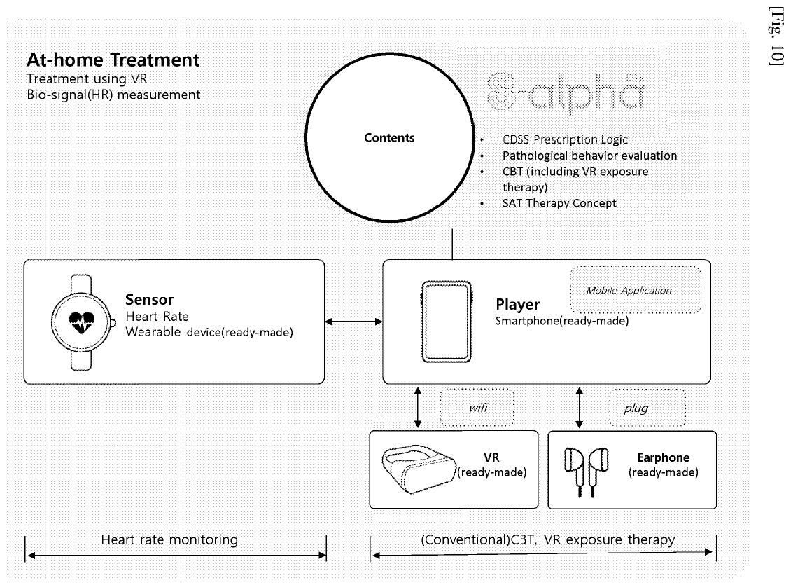

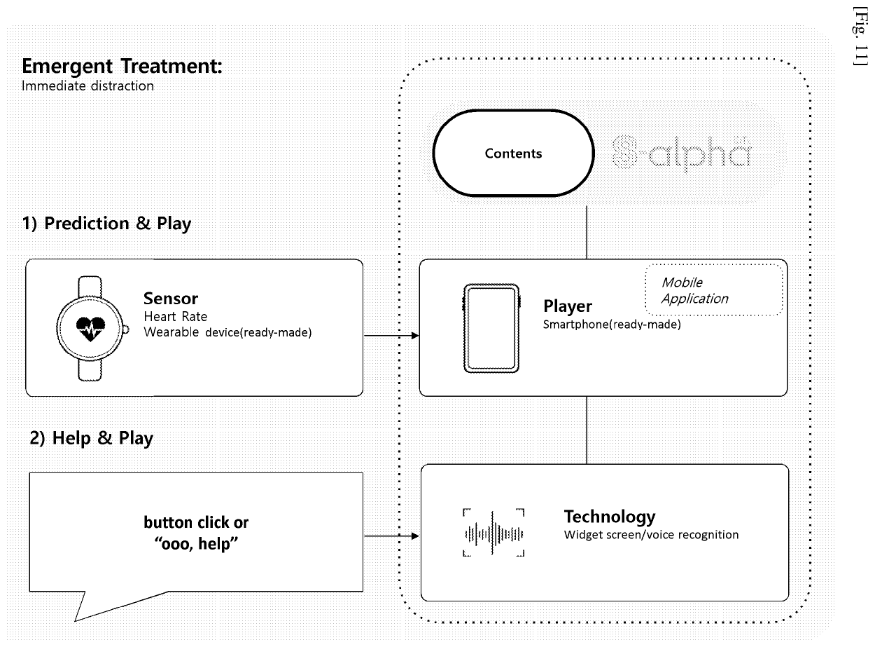

A treatment 870 may be performed. The treatment 870 may include cognitive behavioral therapy (CBT) 880 and/or another therapy 890. The CBT 880 may include a virtual reality (VR) exposure therapy, which may include use of a VR device or any image-displaying device described above. After treatment 870 is performed, a new assessment 810 may be performed to determine the effect of the treatment 870 and to provide feedback to adjust the treatment 870 as necessary. After the new assessment 810 is performed, a new treatment 870 may be performed. The cycle may be repeated as often as needed. The assessment 810 and treatment 870 may both be performed on an electronic device, e.g., a mobile device or computer, and may both be performed on the same device or on separate devices. The VR device may be connected to the electronic device, for example, by a wireless connection, e.g., Wi-Fi, or by a physical connection, e.g., a plug. Associated sound may be provided to the electronic device, for example, by a wireless connection, e.g., Wi-Fi, or by a physical connection, e.g., a plug. The software to run a program for the assessment 810 and treatment 870 may be provided, for example, by an application, e.g., a mobile application. Help may be provided, e.g., by pressing a button on the electronic device or the wearable device, or by speaking a command or prompt. A widget screen and/or voice recognition may be further provided. This will be described further below with reference to FIGS. 10-11.

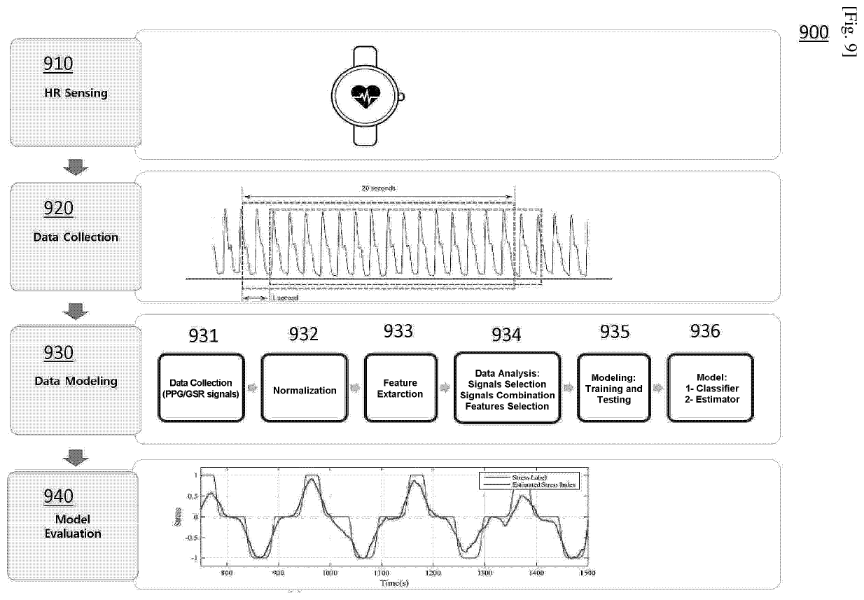

FIG. 9 shows an example process for generating a customized prediction model. The process 900 may include heartrate (HR) sensing 910, data collection 920, data modeling 930, and model evaluation 940. The HR sensing 910 may be performed intermittently, or may be, for example, performed by a wearable device, such as a smart watch or activity tracker, and may be performed continuously in everyday life, including during treatment and training.

The data collection 920 may be performed, for example, during treatment and training. The data collection 920 may include, for example, recording the time of a panic attack, and epoching before and after the panic attack. The data modeling may be saved to a server. The data modeling 930 may include, for example, receiving photoplethysmography (PPG)/galvanic skin response (GSR) signals from the data collection 931, data normalization 932, feature extraction 933, data analysis 934, modeling 935, and model generation 936. The data analysis 934 may include, for example, signal selection, signal combination, and feature selection. The modeling 935 may include, for example, training and testing. The model generation 936 may include a classifier and an estimator. Data modeling may include data from a specified period of time, e.g., from a cohort study. The model evaluation 940 may include both evaluation and refinement of the model.

FIG. 10 shows an example system for at-home treatment. A system 1000 in FIG. 10 may provide an on-site responsive panic disorder digital treatment service. In case a panic disorder occurs in the patient in the real field, e.g., outside of a medical setting, a digital treatment service suitable for the current stage of panic disorder may be provided. The panic disorder may be detected by receiving bio-signals through a biosensor 1010, e.g., a heartrate sensor, such as a photoplethysmogram (PPG), or through user voice and/or screen input, e.g., a widget screen, on a player device 1020. The biosensor 1010 may include, for example, a wearable device, such as a smart watch or activity tracker, which may be custom made for the system 1000, or may be an off-the-shelf device. Bio-signal data from the biosensor 1010 may be sent to the player device 1020. The player device 1020 may include, for example, a mobile device, e.g., a smartphone, and may provide access to the system 1000 via a software application 1030 on the player device 1020, for example, a mobile application. The player device 1020 may be custom made for the system 1000, or may be an off-the-shelf device. The player device 1020 may provide, for example, cognitive behavioral therapy (CBT) and/or VR exposure therapy to the patient. The player device 1020 may use clinical decision support system (CDSS) logic in determining appropriate therapies, based on the bio-signal data from the biosensor 1010. The therapy may include providing contents 1005, which may be viewed on a screen of the player device 1020.

A VR device 1040, e.g., a VR headset, may be provided to provide VR exposure therapy to the patient to treat a panic attack or to train the patient to manage or avoid panic attacks. The VR device 1040 may be connected to the player device 1020 by a VR connection 1045 for transmitting VR image data to the VR device 1040. The VR connection 1045 may be, for example, a wireless connection, e.g., Wi-Fi, or a physical connection, e.g., a plug. Associated sound may be provided from the player device 1020 to a sound output 1050, e.g., headphones, earphones, or one or more external speakers, by a sound connection 1055. The sound connection 1055 may be, for example, a wireless connection, e.g., Wi-Fi or Bluetooth, or a physical connection, e.g., a plug. The sound output 1050 may be separate from or integrated with the VR device 1040. The VR device 1040 and the sound output 1050 may each be either custom-made for the system 1000, or may be off-the-shelf products.

FIG. 11 shows an example system for emergent treatment. FIG. 11 includes similar features as described above in FIG. 10, and a description of repeated elements will be shortened or omitted. A system 1100 may provide immediate distraction as treatment for a panic attack in a patient. In a prediction and play operation 1110, a biosensor, e.g., the biosensor 1010 of FIG. 10, may send bio-signal data to a player device, e.g., the player device 1020 of FIG. 10, which may predict that a panic attack is occurring or about to occur, and may provide therapies as discussed above. In a help and play operation 1120, the patient may directly request help or therapy to be provided by the player device, e.g., a by pressing a button on the biosensor or the player device, or by speaking a command or prompt, for example, “ooo” or “help.” In some embodiments, a widget screen and/or voice recognition technology 1130 may be further provided in the player device or the biosensor to receive a direct input from the user to request help or therapy to be provided by the player device.

FIG. 12 shows an example system for treatment during surgery. FIG. 12 includes similar features as described above in FIGS. 5-7, and a description of repeated elements will be shortened or omitted. In a system 1200 for treatment during surgery, a foot controller 1210 may be provided for the operator 730 to control the control device 732. The control device 732 may be provided with, for example, cognitive therapy software or emotional disorder treatment software for treatment of anxiety or panic in the patient. FIG. 12 shows an example of an image sent to the display 760 of FIG. 7 or the displays 540, 640 of FIGS. 5-6.

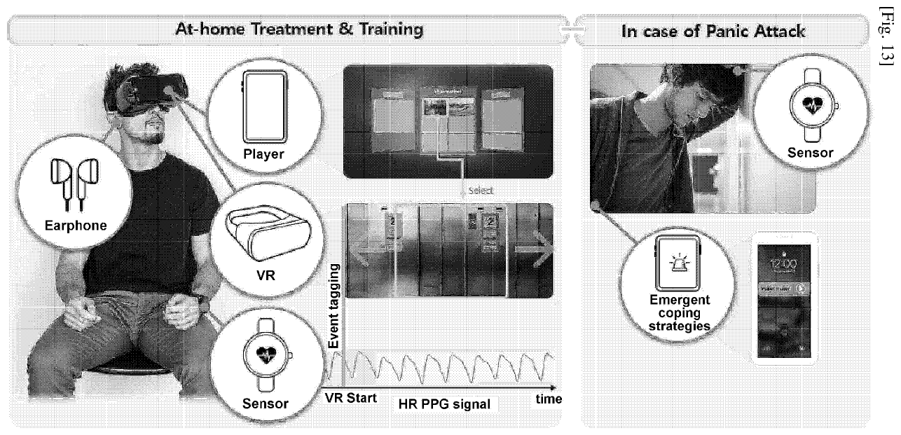

FIG. 13 shows an example system for treatment and training for anxiety or a panic attack. FIG. 12 includes similar features as described above in FIGS. 10-11, and a description of repeated elements will be shortened or omitted. In a system 1300 for treatment and training for anxiety or a panic attack, e.g., at home or outside of a medical setting, a patient may undergo training and treatment for anxiety and panic attacks, for example, using the systems 800, 1000, and 1100 respectively described above with reference to FIGS. 8, 10, and 11. For example, in a training and treatment process 1305, the patient may select a VR therapy 1310, e.g., from a menu or list of VR programs on a player device, e.g., the player device 1020 of FIG. 10, which will be provided to a VR device, e.g., the VR device 1040 of FIG. 10. Sound may be provided to a sound output, e.g., the sound output 1050 of FIG. 10, which may be separate from or integrated with the VR device. The effects of the training and treatment may be monitored, e.g., via a heartrate (HR) PPG signal, for example, by a biosensor, such as the biosensor 1010 of FIG. 10.

As another example, in the system 1300, during a panic attack process 1320, the patient may be provided with one or more emergent coping strategies 1325 as immediate therapy while experiencing a panic attack, which may be provided on the player device, either directly, or via the VR device. The panic attack may be detected, for example, as discussed above with regard to FIG. 11. For example, in some embodiments, the panic attack may be detected using bio-signal data from the biosensor 1010.

The example systems of FIGS. 10-13 may use the mobile device used in the FIG. 9 process 900, and may use the sensor in the wearable device, activity tracker, or external device described above with regard to FIG. 9.

### Computing System

In some aspects, the present disclosure provides a computing system for mitigating emotional or physical tension in a subject in need thereof. In some embodiments, the present disclosure provides a computing system for mitigating emotional or physical tension in a subject during a medical procedure involving a first eye of the subject. In some embodiments, the computing system comprises a display. In some embodiments, the display is configured to provide a first unilateral visual input to a second eye of the subject. In some embodiments, the computing system comprises a sensor. In some embodiments, the sensor is configured to sense the emotional or physical tension in the subject. In some embodiments, the computing system comprises a visual input generation. In some embodiments the visual input generation unit is configured to provide to the subject, using the display, one or more of (i) an image, (ii) a video, (iii) one or more instructions from a healthcare provider associated with the medical procedure for the subject to follow, and (iv) information associated with the medical procedure.

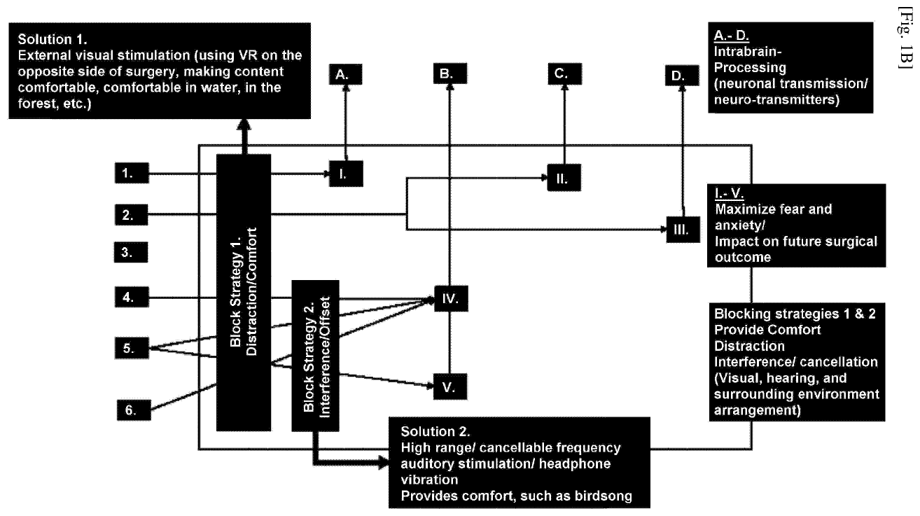

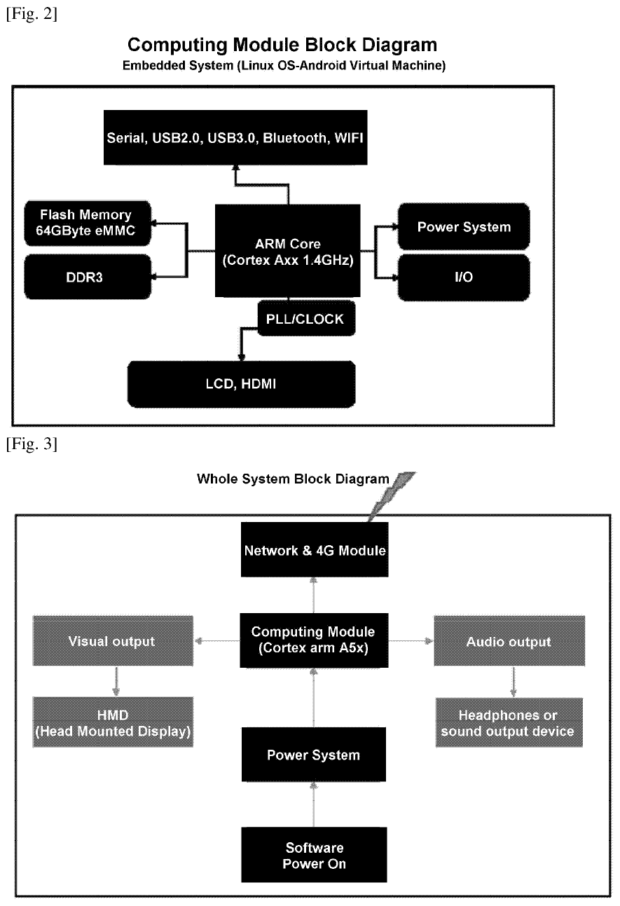

Any of the computing systems mentioned herein can utilize any suitable number of subsystems. In some embodiments, a computing system includes a single computer apparatus, where the subsystems can be the components of the computer apparatus. In other embodiments, a computing system can include multiple computer apparatuses, each being a subsystem, with internal components. A computing system can include desktop and laptop computers, tablets, mobile phones and other mobile devices. FIG. 2 is a block diagram of an exemplary computing module of the present disclosure for use in mitigating emotional or physical tension in a subject during a medical procedure. FIG. 3 is a block diagram of an exemplary system of the present disclosure for use in mitigating emotional or physical tension in a subject during a medical procedure.

The subsystems can be interconnected via a system bus. Additional subsystems include a printer, keyboard, storage device(s), and monitor, which is coupled to display adapter. Peripherals and input/output (I/O) devices, which couple to I/O controller, can be connected to the computing system by any number of connections known in the art such as an input/output (I/O) port (e.g., USB, FireWire®). For example, an I/O port or external interface (e.g., Ethernet, Wi-Fi, etc.) can be used to connect computing system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computing system can include a plurality of the same components or subsystems, e.g., connected together by external interface or by an internal interface. In some embodiments, computing systems, subsystem, or apparatuses can communicate over a network. In such instances, one computer can be considered a client and another computer a server, where each can be part of a same computing system. A client and a server can each include multiple systems, subsystems, or components.

Aspects of embodiments can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

In some aspects, the present disclosure provides a non-transitory computer readable medium having stored thereon software instructions for mitigating emotional or physical tension in a subject during a medical procedure involving a first eye of the subject that, when executed by a processor, cause the processor to provide to the subject, by an electronic device, a first unilateral visual input to a second eye of the subject. In some aspects, the present disclosure provides a non-transitory computer readable medium having stored thereon software instructions for mitigating emotional or physical tension in a subject during a medical procedure involving a first eye of the subject that, when executed by a processor, cause the processor to sense, by a sensor in the electronic device, the emotional or physical tension in the subject. In some aspects, the present disclosure provides a non-transitory computer readable medium having stored thereon software instructions for mitigating emotional or physical tension in a subject during a medical procedure involving a first eye of the subject that, when executed by a processor, cause the processor to, based on the emotional or physical tension in the subject, provide a second unilateral visual input to the second eye of the subject.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computing system), and can be present on or within different computer products within a system or network. A computing system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

The network may comprise any combination of local area and/or wide area networks using both wireless and/or wired communication systems. For example, the network may include the Internet, as well as mobile telephone networks. In one embodiment, the network uses standard communications technologies and/or protocols. Hence, the network may include links using technologies such as Ethernet, 802.11, worldwide interoperability for microwave access (WiMAX), 2G/3G/4G/5G mobile communications protocols (or any other communication protocols that can be an improvement or extension upon the currently available mobile communication protocol), asynchronous transfer mode (ATM), InfiniBand, PCI Express Advanced Switching, etc. Other networking protocols used on the network can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), real time streaming protocol (RTSP), and the like. The data exchanged over the network can be represented using technologies and/or formats including image data in binary form (e.g., Portable Networks Graphics (PNG)), the hypertext markup language (HTML), video formats, the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

In some embodiments the electronic device may include a central processing unit (computing module). The control module may communicate with the server via the communication network to transmit control instructions from the healthcare provider for providing a unilateral visual input to the subject.

The computing module can execute a sequence of machine-readable instructions, which can be embodied in a program or software. The instructions may be stored in a memory location, such as the memory. The instructions can be directed to the computing module, which can subsequently program or otherwise configure the computing module to implement methods of the present disclosure. Examples of operations performed by the computing module can include fetch, decode, execute, and writeback. The computing module can be part of a circuit, such as an integrated circuit. One or more other components of the system can be included in the circuit. In some cases, the circuit is an application specific integrated circuit (ASIC).

A storage unit may be used to store files, such as drivers, libraries and saved programs. The storage unit can store user data, e.g., user preferences and user programs. The electronic device in some cases can include one or more additional data storage units that are external to the electronic device, such as located on a remote server that is in communication with the electronic device through an intranet or the Internet.

The electronic device can communicate with one or more remote computer systems through the network. For instance, the electronic device can communicate with a remote computer system of a user or other immersive display devices (e.g., a head-mounted display or HMD). Examples of remote computer systems include personal computers (e.g., portable PC), slate or tablet PCs (e.g., Apple® iPad, Samsung® Galaxy Tab), telephones, Smart phones (e.g., Apple® iphone, Android-enabled device, Blackberry®), personal digital assistants, Oculus Rift, HTC Vive, or other VR/AR systems.

Methods as described herein can be implemented by way of machine (e.g., computer processor) executable code stored on an electronic storage location of the electronic device, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The electronic device may include or be in communication with an electronic display device for providing, for example, the unilateral visual input, selection of types of datasets to be displayed, feedback control command configuration tools, any other features related to the selection, transmission, and display of datasets, or any other features described herein. In some embodiments, the display device may include an HMD, or a pair of virtual reality (VR) or augmented reality (AR) enabled glasses. The display device might not be a part of the electronic device and may be communicatively coupled to the electronic device to receive the unilateral visual input. For example, the display devices may be a projection system (e.g., immersive display system).

In some instances, the display device may comprise a mobile device (e.g., mobile phone) mounted onto a foldable headgear. The mobile device may comprise a graphical display configured to display a first-person-view (FPV) of the environment. In such configuration, the mobile device may also function as an electronic device, and may be communicatively coupled to the network to receive the unilateral visual input. The sensor may also be fully integrated into the mobile device.

The electronic device may include a sensor. The sensor may or may not be part of the electronic device. The sensor may be configured to be communicatively coupled to the electronic device. For example, the sensor may be connected to the electronic device via one or more wireless methods, peer-to-peer methods, or a wired method such as a connection via USB. The sensor may be configured to detect and collect relevant information related to the subject's emotional or physical tension experienced before, during, or after a medical procedure. In other embodiments, the display device (e.g., HMDs) may include a sensor that may be able to detect the user's specific motion, and track, for instance, eye movements.

The sensor may track eye movements by measuring the point of gaze (i.e., where a user may be looking) or the motion of an eye relative to the head. The sensor may comprise an eye tracking device for measuring such eye positions and/or eye movements. Various types of eye tracking devices may be combined.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a WTRU, UE, terminal, base station, RNC, or any host computer.

1. A method of mitigating emotional or physical tension in a subject during a medical procedure involving a first eye of the subject, the method comprising:
providing to the subject, by an electronic device, a first unilateral visual input to a second eye of the subject,
sensing, by a sensor connected to the electronic device, the emotional or physical tension in the subject; and
based on the emotional or physical tension in the subject, providing a second unilateral visual input to the second eye of the subject to mitigate the emotional or physical tension in the subject.

2-309: (canceled)
